(19) United States
(12) Patent Application Publication
(10) Pub. No.: US 2024/0099965 A1
(43) Pub. Date: Mar. 28, 2024

(54) OXYTOCIN READY TO INFUSE DOSAGE FORM

(71) Applicant: Sun Pharmaceutical Industries Limited, MUMBAI (IN)

(72) Inventors: Samarth KUMAR, Baroda, Gujarat (IN); Neeraj Kumar GARG, Baroda, Gujarat (IN); Ajay Jaysingh KHOPADE, Baroda, Gujarat (IN); Subhas Balaram BHOWMICK, Baroda, Gujarat (IN)

(21) Appl. No.: 18/264,067

(22) PCT Filed: Feb. 3, 2022
(86) PCT No.: PCT/IB2022/050956

§ 371 Date: Aug. 2, 2023

(30) Foreign Application Priority Data
Feb. 3, 2021 (IN) 202121004764

(51) Int. Cl. A61K 9/00; A61J 1/14; A61K 9/08; A61K 38/095; A61K 47/12; A61K 47/26; A61K 47/40

(52) U.S. Cl. CPC A61K 9/0029; A61J 1/1468; A61K 9/08; A61K 38/095; A61K 47/12; A61K 47/26; A61K 47/40

(57) ABSTRACT

The present disclosure relates to a ready-to-use or a ready to administer parenteral dosage form of oxytocin or a pharmaceutically acceptable salt thereof comprising a ready-to-infuse, stable aqueous solution of oxytocin or a pharmaceutically acceptable salt thereof. The solution can be administered to a patient in need thereof without manipulations in terms of its concentration and is stable for a prolonged period of time.

### CROSS REFERENCE

The present patent application claims priority from Indian provisional patent application no. 202121004764 filed on Feb. 3, 2021.

### FIELD OF THE INVENTION

The present disclosure relates to a parenteral dosage form of oxytocin or a pharmaceutically acceptable salt thereof comprising a ready-to-infuse, stable aqueous solution of oxytocin or a pharmaceutically acceptable salt thereof. The solution can be administered to a patient in need thereof without manipulations in terms of its concentration and is stable for a prolonged period of time.

### BACKGROUND OF THE INVENTION

Oxytocin is a nonapeptide with two cysteine residues that form a disulfide bridge between positions 1 and 6. It is an extremely short-lived, fast acting hormone, made by the hypothalamus of the brain, stored in the posterior pituitary, and released into the blood as needed. It stimulates certain smooth muscle cells, constricts certain blood vessels, and facilitates the sensitivity of some tissues to other hormones and nerves. The hormone is prepared synthetically to avoid possible contamination with vasopressin (ADH) and other small polypeptides with biological activity. Oxytocin has the empirical formula C₄₃H₆₆N₁₂O₁₂S₂. The structural formula is as follows:

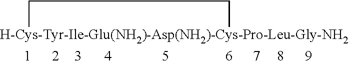

Oxytocin has special effects on uterine muscle contractions in both birth and orgasm, the vascular constriction that lessens placental separation bleeding, and the let-down reflex that nursing mothers have when babies cry. According to the World Health Report 2005 as issued by the World Health Organization, in Africa, Asia and Latin America each year half a million women die as a result of problems during pregnancy and childbirth. In Africa and Asia, at least 25% of those deaths can be attributed to hemorrhages, most commonly caused by failure of the uterus to contract adequately after childbirth (atonicity). This makes hemorrhaging the leading cause of maternal deaths in these continents (Khan et al., Lancet 367 (9516): 1066-1074, 2006). In addition to this, in third world countries, it is often practically and/or economically impossible to protect pharmaceutical preparations like oxytocin from the harmful effects of high temperatures during transportation, storage and use. As it is degraded in the gastrointestinal tract, oxytocin is typically administered as a liquid formulation by injection or as a nasal spray.

Oxytocin is currently indicated for stimulation of uterine contraction to induce labor, for the control of postpartum hemorrhage (PPH) following delivery of the placenta and for stimulation of lactation for breast-feeding. Currently, oxytocin is available in the United States as a single use 1 ml and multidose 10 ml and 50 ml glass vial. The formulation is typically present at a concentration of 10 USP Units/ml. The available formulation contains chlorobutanol at 0.5% (5 mg/ml) as a preservative.

The main method of administration of oxytocin for the induction or stimulation of labor is through intravenous (IV) infusion. To prepare the usual solution for infusion, 1 mL Oxytocin Injection, 10 USP Units/mL is combined aseptically with 1000 mL of non-hydrating diluent (i.e., physiologic electrolyte solution) resulting in an oxytocin concentration of 1 USP Unit/100 mL. The combined solution is then rotated in the infusion bottle to ensure thorough mixing. The infusion bottle typically contains 10 mU/mL. Then, this dilute oxytocin solution is delivered to a patient through use of a constant infusion pump or another similar device to accurately control the rate of infusion.

A major drawback of existing oxytocin formulations is that they require multiple mixing steps prior to being administered to a patient, thus increasing the risk of errors, and possible aseptic methods leading to infections. Furthermore, the container volume for the diluted oxytocin formulation is typically large, about 1,000 mL, however not more than 250 mL of oxytocin solution fluid is used for medically induced labor. This means that a major portion of the diluted oxytocin formulation is currently being wasted. Additionally the use of chlorobutanol as a preservative in the existing formulation is a major drawback as chlorobutanol is a skin irritant and is also highly toxic to the liver. Thus, it would be desirable to avoid chlorobutanol and other such preservatives in oxytocin formulations.

Further, concerns are frequently raised about the challenge of maintaining the effectiveness of oxytocin for PPH prevention in tropical settings when refrigerated storage conditions cannot be assured. When oxytocin is exposed to high temperatures for extended periods of time, the effective dose can drop below the prescribed industry standard of 90 to 110% of the labeled claim. Further drug degradation has safety and efficacy implications, and the degradation of the active ingredient can lead to the formation of toxic decomposition products. However, as a peptide, the degradation products of oxytocin are not believed to be toxic. Therefore, while degradation may not necessarily be a safety issue, specifically in the case of oxytocin (Hodgins and Lukulay, Int J Gynaecol Obstet. 2017, 136(3):253-254), it can still lead to a reduced active pharmaceutical ingredient level and hence compromised performance. Further the review literature revisiting the Stability and Storage Specifications of Oxytocin Injection (USAID, July 2018), provides insight on assay drops in various marketed oxytocin formulations worldwide. The pharmacopoeia limits of assay in the range of 90-110% as accepted by various standard is critical to meet for such formulation. Furthermore, dilute solutions of oxytocin generally have a significantly shorter shelf-life than concentrated oxytocin solutions.

Thus, there is a need for a stable parenteral dosage form of oxytocin, which comprises an aqueous solution of Oxytocin that is ready-to-infuse and can be administered without any manipulation, i.e., in the pre-diluted form that can be directly infused or injected thus eliminating the risk of any potential calculation or dilution error as well as risk of microbiological contamination during handling and that at the same time is stable for a prolonged period of time.

### SUMMARY OF THE INVENTION

The present disclosure provides a parenteral dosage form comprising a ready-to-infuse, stable aqueous solution comprising oxytocin or its pharmaceutically acceptable salt.

The present disclosure relates to an aqueous solution of oxytocin that is ready-to-infuse without any manipulation, i.e. in the pre-diluted form that can be directly infused or injected thus eliminating the risk of any potential calculation or dilution error as well as risk of microbiological contamination during handling and at the same time is stable for prolonged periods of time. More particularly, a stable, ready-to-infuse, aqueous parenteral dosage form is provided, comprising oxytocin or its pharmaceutically acceptable salt, a pH adjusting agent to provide a pH in the range of 3.0 to 5.0, and at least one disaccharide.

One embodiment is a ready-to-infuse parenteral dosage form comprising a stable aqueous solution having a pH of 3.0 to 5.0 comprising:
(a) oxytocin or a pharmaceutical acceptable salt thereof in a concentration range of about 0.01 to about 10 IU per mL;
(b) a disaccharide;
(c) sodium acetate; and
(d) acetic acid/sodium hydroxide,
   wherein (i) the solution is substantially free of chlorobutanol and (ii) the oxytocin concentration, after storage at 2-8° C., is 90% or greater than the oxytocin concentration prior to storage. In one preferred embodiment, the level of any single impurity in the solution is less than 3% by weight of the amount of oxytocin present prior to storage after storage at 2-8° C. The solution is preferably substantially free of divalent metal salts.

In another embodiment of the present disclosure, the ready-to-infuse parenteral dosage form comprises a stable aqueous solution having a pH of 3.0 to 5.0 comprising:
(a) oxytocin or a pharmaceutical acceptable salt thereof in a concentration range of about 0.01 to about 10 IU per mL;
(b) a disaccharide;
(c) sodium acetate; and
(d) acetic acid/sodium hydroxide,
   wherein (i) optionally the solution is substantially free of sodium chloride and dextrose and (ii) the oxytocin concentration, after storage at 2-8° C., is 90% or greater than the oxytocin concentration prior to storage. In one preferred embodiment, the level of any single impurity in the solution is less than 3% by weight of the amount of oxytocin present prior to storage after storage at 2-8° C. The solution is optionally free of EDTA and salts thereof.

In one embodiment of the present disclosure, the ready-to-infuse parenteral dosage form comprises a stable aqueous solution having a pH of 3.0 to 5.0 comprising oxytocin or a pharmaceutical acceptable salt thereof in a concentration range of about 0.01 to about 10 IU per mL; sucrose; sodium acetate; and acetic acid/sodium hydroxide, wherein (i) the solution is substantially free of chlorobutanol and (ii) the oxytocin concentration, after storage at 2-8° C., is 90% or greater than the oxytocin concentration prior to storage. In one preferred embodiment, the level of any single impurity in the solution is less than 3% by weight of the amount of oxytocin present prior to storage after storage at 2-8° C.

In yet another embodiment of the present disclosure, the ready-to-infuse parenteral dosage form comprises a stable aqueous solution having a pH of 3.0 to 5.0 comprising: oxytocin or a pharmaceutical acceptable salt thereof in a concentration range of about 0.01 to about 10 IU per mL; sucrose; mannitol; sodium acetate; and acetic acid/sodium hydroxide, wherein (i) the solution is substantially free of chlorobutanol and (ii) the oxytocin concentration, after storage at 2-8° C., is 90% or greater than the oxytocin concentration prior to storage. In one preferred embodiment, the level of any single impurity in the solution is less than 3% by weight of the amount of oxytocin present prior to storage after storage at 2-8° C.

In some embodiment of the present disclosure, the ready-to-infuse parenteral dosage form comprises a stable aqueous solution having a pH of 3.0 to 5.0 comprising: oxytocin or a pharmaceutical acceptable salt thereof in a concentration range of about 0.01 to about 10 IU per mL; mannitol; Hydroxypropyl Betadex (HPBCD or HP-β-CD); sodium acetate; and acetic acid/sodium hydroxide, wherein (i) the solution is substantially free of chlorobutanol and (ii) the oxytocin concentration, after storage at 2-8° C., is 90% or greater than the oxytocin concentration prior to storage. In one preferred embodiment, the level of any single impurity in the solution is less than 3% by weight of the amount of oxytocin present prior to storage after storage at 2-8° C.

In some other embodiment of the present disclosure, the ready-to-infuse parenteral dosage form comprises a stable aqueous solution having a pH of 3.0 to 5.0 comprising: oxytocin or a pharmaceutical acceptable salt thereof in a concentration range of about 0.01 to about 10 IU per mL; sodium chloride; Hydroxypropyl Betadex (HPBCD or HP-β-CD); sodium acetate; and acetic acid/sodium hydroxide, wherein (i) the solution is substantially free of chlorobutanol and (ii) the oxytocin concentration, after storage at 2-8° C., is 90% or greater than the oxytocin concentration prior to storage. In one preferred embodiment, the level of any single impurity in the solution is less than 3% by weight of the amount of oxytocin present prior to storage after storage at 2-8° C.

### DETAILED DESCRIPTION OF THE INVENTION

As used herein, the word “a” or “plurality” before a noun represents one or more of the particular noun.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

The term “substantially free” refers to a composition containing less than 0.01% by weight (for example, less than 0.005% or 0.001% by weight) of the specified component.

The term “dosage form” refers to a pharmaceutical composition or a pharmaceutical formulation and can be used interchangeably to refer to a dosage form.

Unless otherwise defined, all technical and scientific terms used herein have the same meaning as commonly understood by one of ordinary skill in the art to which this invention belongs.

Methods and materials are described herein for use in the present invention; other, suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

Suitable disaccharides include, but are not limited to, sucrose, lactulose, lactose, maltose, trehalose, cellobiose, kojibiose, nigerose, isomaltose, sophorose, laminarbiose, gentiobiose, turanose, maltulose, palatinose, gentiobiulose, mannobiose, melibiose, melibulose, rutinose, rutinulose and xylobiose.

In another embodiment, the present disclosure relates to a stable, ready-to-infuse, aqueous parenteral dosage form comprising: (i) an aqueous solution comprising oxytocin or its pharmaceutically acceptable salt, a pH adjusting agent to provide a pH in the range of 3.0 to 5.0, at least one disaccharide, and an osmogen; and (ii) an infusion container filled with the aqueous solution.

The term ‘ready-to-infuse’ as used herein means that the aqueous drug solution is sterile and suitable for direct intravenous infusion or injection without manipulation, that is, no intermediate steps of dilution, reconstitution, dispensing, sterilization, transfer, handling or compounding are required before administration or infusion of the drug solution to the patient. The aqueous drug solution can be directly administered parenterally from the container of the dosage form. The term “ready-to-infuse” is synonymous with “ready-to-inject” or “ready-to-administer” or “directly administering” or “direct intravenous infusion” or “direct delivery” or “ready to use”. The ready-to-infuse parenteral dosage form according to the present invention avoids the inconvenience of reconstituting or diluting a lyophilized or concentrated parenteral formulation into infusion diluents prior to infusion, as well as the risk of any potential calculation or dilution error and microbiological contamination during handling. Also a ready-to-infuse parenteral dosage form can be said to be a premixed dosage form which can be administered directly without any dilution or mixing requirement. Moreover, oxytocin is sometimes delivered in emergency situation viz. for induction of labor, to control PPH and stimulation or reinforcement of labor state emergency medicine and hence reduced time in getting the dose ready in ready-to-infuse dosage forms is advantageous and preferred. The present disclosure provides an improved stable parenteral dosage form having a ready-to-infuse aqueous solution of oxytocin. The parenteral dosage form is not a semi-solid topical dosage form (such as gel, hydrogel, emulgel, paste, cream, ointment etc.) or non-aqueous dosage form that is not suitable for parenteral administration.

The parenteral dosage form disclosed in the present invention is ‘stable’. As used herein, the term ‘stable’ means that the dosage form does not physically change and about 80 to about 120 percent of oxytocin remains after storage under refrigerated conditions (2-8° C.) for at least a prolonged period of time such as for at least 3 months, preferably 6 months to 12 months, preferably 18 months to 24 months. The solution is also stable, when stored at 25° C. (room temperature condition) for a period of at least 3 months, preferably 6 months, more preferably 9 months to 12 months. When stored at these conditions, the aqueous solution of oxytocin or its pharmaceutically acceptable salt remains chemically stable, wherein various parameters such a drug content (assay of oxytocin) and content of related substances, i.e. known impurities, unknown impurities and total impurity, remains within specified limits. Suitably the assay of oxytocin remains within 90%-110% by weight of the label claim, the content of total impurities remains within 16% by weight and the content of any single known impurity remains within 3% by weight (relative to the label claim for the oxytocin content or the actual oxytocin content prior to storage). The assay may be determined using an HPLC technique.

The aqueous solution of oxytocin according to the present disclosure comprises at least one osmogen.

The term ‘osmogen’ as used herein can be interchangeably used with the term ‘tonicity adjusting agent’ or ‘osmotic agent’.

In one embodiment, non-limiting examples of osmogen used in the aqueous solution of the present disclosure may be selected from sodium chloride, calcium chloride, magnesium chloride, potassium chloride, dextrose, glycerin, glycerol, sucrose, mannitol, xylitol, fructose, mannose, maltitol, inositol or trehalose, other inorganic salts, urea or mixtures thereof.

In another embodiment, non-limiting examples of an osmogen that may be used in the aqueous solution of the present disclosure are selected from sodium chloride, sucrose, mannitol, Hydroxypropyl Betadex (HPBCD or HP-β-CD) or mixtures thereof. In one preferred embodiment, the osmogen in the solution comprises at least 50% by weight of sucrose, based on the total amount of osmogen in the solution. More preferably, the osmogen in the solution comprises at least 50% by weight of sucrose, based on the total amount of osmogen in the solution. In yet another embodiment, the only osmogen in the solution is sucrose. In yet another embodiment, the osmogen in the solution is a combination of sucrose and mannitol (for example, at a weight ratio of 10:1 to 2:1, such as 7:1 to 3:1). In yet another embodiment, the osmogen in the solution is a combination of mannitol and Hydroxypropyl Betadex (HPBCD or HP-β-CD). In yet another embodiment, the osmogen in the solution is a combination of sodium chloride and Hydroxypropyl Betadex (HPBCD or HP-β-CD).

In one embodiment the present invention provides a ready-to-infuse parenteral dosage form comprising a stable aqueous solution having a pH of 3.0 to 5.0 comprising:
(a) oxytocin or a pharmaceutical acceptable salt thereof in a concentration range of about 0.01 to about 10 IU per mL;
(b) an excipient selected from sodium chloride, sucrose, mannitol, Hydroxypropyl Betadex (HPBCD or HP-β-CD) or mixtures thereof, wherein the mixture comprises at least two excipient.
   wherein (i) the solution is substantially free of chlorobutanol and dextrose and (ii) the oxytocin concentration, after storage at 2-8° C., is 90% or greater than the oxytocin concentration prior to storage.

In an embodiment, the parenteral dosage form comprises a ready-to-infuse, stable aqueous solution comprising oxytocin or its pharmaceutically acceptable salt, at least one osmogen, a pH adjusting agent, and at least one sugar moiety. In an aspect of the present invention, the sugar moiety may be a disaccharide, sugar alcohol or a combination thereof.

In another embodiment, the disclosed stable, ready-to-infuse, aqueous parenteral dosage form comprises oxytocin or its pharmaceutically acceptable salt, a pH adjusting agent to provide a pH in the range of about 3.0 to about 5.0, at least one osmogen and a buffer component. In an aspect, the buffer component is sodium acetate.

In a further embodiment, the disclosed stable, ready-to-infuse, aqueous parenteral dosage form comprises oxytocin or its pharmaceutically acceptable salt, a pH adjusting agent to provide a pH in the range of about 3.0 to about 5.0, at least one pharmaceutically acceptable excipient selected from an osmogen, a buffering agent, at least one sugar moiety and an infusion container filled with said aqueous solution.

In one aspect the sugar moiety is selected from at least one disaccharide, a combination of disaccharide with other sugars such as sugar alcohol selected from group of mannitol, sorbitol, xylitol, erythritol and the likes.

In yet another embodiment, the disclosed stable, ready-to-infuse, aqueous parenteral dosage form comprises oxytocin or its pharmaceutically acceptable salt, a pH adjusting agent to provide a pH in the range of about 3.0 to about 5.0, a pharmaceutically acceptable excipient which is sodium chloride, sodium acetate, or sucrose and an infusion container filled with said aqueous solution.

In one embodiment, the disclosed stable, ready-to-infuse, aqueous parenteral dosage form comprises oxytocin or its pharmaceutically acceptable salt, a pH adjusting agent to provide a pH in the range of about 3.0 to about 5.0, at least one osmogen, and a non-alcoholic solvent or diluent.

In one embodiment, the present disclosure provides a parenteral dosage form comprising a ready-to-infuse, stable aqueous solution comprising oxytocin or its pharmaceutical acceptable salt, at least one osmogen, and a disaccharide, wherein the pH of the solution in range of pH 3.0 to 5.0, and the level of any single known impurity is not more than 3%, and the level of total impurities is not more than 16%.

In another embodiment, the present disclosure provides a ready-to-infuse parenteral dosage form comprising a stable aqueous solution comprising: oxytocin or its pharmaceutical acceptable salt thereof, and a disaccharide, wherein the assay % of oxytocin in the solution is at least 90%.

In yet another embodiment, the present disclosure provides a ready-to-infuse parenteral dosage form comprising a stable aqueous solution comprising:
a. oxytocin or its pharmaceutical acceptable salt thereof in a concentration range of about 0.005 to about 10 IU per mL, and
b. a disaccharide,
   wherein the assay % of oxytocin in the solution is at least 90%, and the level of any single known impurity in the solution is less than 3% by weight when stored at 2-8° C. and/or 25° C./40% RH.

In another embodiment, the present disclosure provides a ready-to-infuse parenteral dosage form comprising a stable aqueous solution comprising:
a. oxytocin or its pharmaceutical acceptable salt thereof in a concentration range of about 0.005 to about 10 IU per mL, and
b. a disaccharide and sugar alcohol,
   wherein the assay % of oxytocin in the solution is at least 90%, and the level of any single known impurity in the solution is less than 3% by weight when stored at 2-8° C. and/or 25° C./40% RH.

In another aspect, the ready-to-infuse parenteral dosage form according to the present disclosure, further comprises at least one osmogen. In another aspect, the pH of the aqueous solution is in the range of about pH 3.0 to about pH 5.0.

In another aspect, in the ready-to-infuse parenteral dosage form according to the present disclosure, the level of any single known impurity in the solution is less than 3% by weight when stored at 2-8° C. and/or 25° C./40% RH. In yet another aspect, in the ready-to-infuse parenteral dosage form according to the present disclosure, the level of total impurities in the solution is less than 16% by weight when stored at 2-8° C. and/or 25° C./40% RH.

In one aspect, the ready-to-infuse parenteral dosage form according to the present disclosure has an assay of oxytocin in the solution within 90% to 110%.

In an aspect, the ready-to-infuse parenteral dosage form according to the present disclosure comprises at least one osmogen that is selected from the group consisting of sodium chloride, calcium chloride, magnesium chloride, potassium chloride, sugars selected from dextrose, glycerin, glycerol, sucrose, mannitol, xylitol, fructose, mannose, maltitol, inositol or trehalose; other inorganic salts, urea or a mixture thereof. In an aspect, said sugar is selected from a monosaccharide, disaccharide, polysaccharide or sugar alcohol or a combination thereof. In an aspect, the sugar is a combination of sucrose and mannitol.

In another aspect, the ready-to-infuse parenteral dosage form according to the present disclosure comprises a disaccharide selected from sucrose or a combination of saccharides, selected from sucrose, lactulose, lactose, maltose, trehalose, cellobiose, kojibiose, nigerose, isomaltose, sophorose, laminarbiose, gentiobiose, turanose, maltulose, palatinose, gentiobiulose, mannobiose, melibiose, melibulose, rutinose, rutinulose or xylobiose.

In one embodiment, the present disclosure provides a parenteral dosage form comprising a ready-to-infuse, stable aqueous solution comprising oxytocin or its pharmaceutical acceptable salt in a concentration range of about 0.005 IU per ml to about 10 IU per ml, and at least one osmogen in a concentration range of about 1 mg/mL to about 110 mg/mL, wherein the pH of the solution is in range of pH 3.0 to 5.0, said aqueous solution is stable upon storage at 2-8° C. for at least 6 months, the level of any single known impurity is not more than 3% by weight of active, preferably less than 2% by weight of active, and the total impurities are less than 10% by weight of active, preferably less than 5% by weight of active.

In an embodiment, the ready-to-infuse parenteral dosage form according to the present disclosure, is stable upon storage at 25° C./40% RH for at least 6 months, the level of any single known impurity is not more than 3% by weight of active and the total impurities are less than 16% by weight of active, preferably less than 8% by weight of active.

In another embodiment, the parenteral dosage form according to the present disclosure comprises an aqueous solution comprising oxytocin or its pharmaceutical acceptable salt in a concentration range of about 0.005 to about 10 IU per mL, at least one osmogen in a concentration range of about 1 to about 110 mg/mL, and sucrose in a concentration range of about 5 to about 90 mg/mL, wherein the pH of the solution is in range of pH 3.0 to 5.0. In an aspect, the parenteral dosage form has an assay of oxytocin within 90-110% for a storage period of at least 6 months at room temperature and/or 2-8° C.

In yet another aspect, the present disclosure provides a use of a stable parenteral dosage form for induction of labor in patients with a medical indication for the initiation of labor selected from Rh problems, maternal diabetes, and/or preeclampsia.

In yet another embodiment, the present disclosure provides use of a disaccharide for preparation of a ready-to-infuse, stable aqueous solution of oxytocin or its pharmaceutical acceptable salt, wherein in the solution, the level of any single known impurity is not more than 3% when stored at 2-8° C. and/or at 25° C./40% RH.

In one embodiment, the present disclosure provides a parenteral dosage form comprising a ready-to-infuse, stable aqueous solution comprising oxytocin or its pharmaceutical acceptable salt in a concentration range of about 0.01 IU per ml to about 10 IU per ml, at least one osmogen in a concentration range of about 1 mg/mL to about 110 mg/mL, sucrose in a concentration range of about 5 mg/mL to about 90 mg/mL, wherein the pH of the solution is in the range of pH 3.0 to 5.0, said aqueous solution is stable upon storage at 2-8° C. for at least 6 months, the level of any single known impurity is not more than 3% by weight of active, preferably less than 2% by weight of active, and the total impurities are less than 10% by weight of active, preferably less than 5% by weight of active. In an aspect osmogen is selected from mannitol, sucrose, sodium chloride, Hydroxypropyl Betadex (HPBCD or HP-β-CD), or a combination thereof comprising at least two or more osmogen.

In an embodiment, the ready-to-infuse parenteral dosage form according to the present disclosure, is stable upon storage at 25° C./40% RH for at least 6 months, the level of any single known impurity is not more than 3% by weight of active and the total impurities are less than 16% by weight of active, preferably less than 8% by weight of active.

In some aspects of the above embodiments, the disclosed parenteral dosage form prevents a drop in assay of oxytocin below 90% for a longer period of time at least 6 months, preferably for 12 or 18 months.

In one embodiment, said aqueous solution comprising oxytocin or its pharmaceutically acceptable salt as the active ingredient is filled into a infusion container. Oxytocin or its pharmaceutically acceptable salt is present in the aqueous solution at a concentration which allows direct infusion of the aqueous solution to the patient without the need of further dilution. For example, the oxytocin or its pharmaceutically acceptable salt may be present at a concentration ranging from about 0.005 IU per mL to about 10 IU per mL, and in some embodiments from about 0.006 IU/mL to about 10 IU/mL, such as, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.5, 2.8, 3.0, 3.4, 4.0, 4.5, 5.0, 5.5, 6.0, 6.5, 7.0, 7.5, 8.0, 8.5, 9.0, 9.5 or 10 IU/mL. In yet further embodiments, oxytocin or its pharmaceutically acceptable salt may be present in the aqueous solution in an amount from about 0.05 IU/mL to about 5.0 IU/mL. In one particular embodiment, oxytocin or its pharmaceutically acceptable salt may be present in the aqueous solution in an amount of about 0.06 IU/mL.

In one embodiment according to the present disclosure, the assay of oxytocin or its pharmaceutically acceptable salt in the dosage form is within 90-110% for a prolonged period of time, such as for at least 6 months, preferably 12 months, more preferably 18 months to 24 months.

In some embodiments, the disclosed parenteral dosage form includes an aqueous solution of oxytocin or its pharmaceutically acceptable salt filled into an infusion container, which may be rigid or flexible in nature. The volume capacity of each unit of the container may range from about 50 ml to about 1000 ml. The aqueous solution may be present in the infusion containers in volumes ranging from about 50 ml to about 1000 ml per infusion container, for example, 50 ml, 75 ml, 100 ml, 120 ml, 125 ml, 140 ml, 150 ml, 160 ml, 175 ml, 180 ml, 190 ml, 200 ml, 220 ml, 225 ml, 240 ml, 250 ml, 260 ml, 275 ml, 280 ml, 290 ml, 300 ml, 320 ml, 325 ml, 340 ml, 350 ml, 360 ml, 375 ml, 380 ml, 390 ml, 400 ml, 420 ml, 425 ml, 430 ml, 440 ml, 450 ml, 460 ml, 470 ml, 475 ml, 480 ml, 490 ml, 500 ml, 550 ml, 600 ml, 650 ml, 700 ml, 750 ml, 800 ml, 850 ml, 900 ml, 950 ml or 1000 ml. In some embodiments, the ready-to-infuse parenteral dosage form is filled in a large volume container, such as an infusion bag, which can accommodate a volume of at least 50 ml, including volumes from about 100 ml to about 500 ml.

In one embodiment, the at least one osmogen is present at a concentration ranging from about 1 mg/mL to about 110 mg/mL. In a further embodiment, the at least one osmogen is present at a concentration ranging from about 3 mg/mL to about 85 mg/mL. In a yet further embodiment, the concentration of the at least one osmogen is from about 5 mg/mL to about 75 mg/mL.

In one embodiment, the at least one osmogen is present at a concentration of about 5-95 mg/mL. In another embodiment, the osmogen can be a mixture of two or more osmogens, and at least one of the osmogen is a sugar moiety.

In one embodiment, the disclosed stable parenteral dosage form may optionally comprise an organic solvent. In a further embodiment, the organic solvent may be present in the aqueous solution in an amount ranging from about 1.0% w/v to about 50% w/v, from about 1.0% w/v to about 20.0% w/v, or from about 1.0% w/v to about 15.0% w/v, for example, 1.0, 1.2, 1.3, 1.4, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5 or 15% w/v. In one embodiment, an organic solvent is present in the disclosed ready-to-infuse aqueous solution in an amount ranging from about 1.0% w/v to about 5.0% w/v, and in certain embodiments, about 2-3% w/v.

In a further embodiment, the disclosed parenteral dosage form or the ready-to-infuse aqueous solution may further comprise other parentally acceptable excipients. Examples of parentally acceptable excipients that may be used include, but are not limited to, pH adjusting agents and buffers, osmogens or osmotic/tonicity adjusting agents, chelating agents, and the like. In one embodiment, the dosage form is free of chlorobutanol and preservatives. In another embodiment the dosage form may optionally comprise one or more anti-oxidants.

In one embodiment, the aqueous solution is free of one or more of chlorobutanol, and a divalent metal salt. In another embodiment, the aqueous solution is free of chlorobutanol. In yet another embodiment, the aqueous solution is free of divalent metal salts.

In one embodiment, the aqueous solution is optionally free of one or more of sodium chloride, dextrose, EDTA and salts thereof. In another embodiment, the aqueous solution is free of sodium chloride, and dextrose. In one embodiment, the aqueous solution is free of one or more of dextrose, EDTA and salts thereof. In yet another embodiment, the aqueous solution is free of chlorobutanol, sodium chloride, dextrose, a divalent metal salt, and EDTA and salts thereof.

The disclosed parenteral dosage form may optionally comprise other components, including, for example, and without limitation, Butylated Hydroxyanisole, Butylated Hydroxytoluene, Ammonium Sulphate, Sodium Metabisulfite, Edetate Disodium, Hydroxypropyl Betadex (HPBCD or HP-β-CD), L-Methionine, Potassium Phosphate Monobasic, Anhydrous Lactose, Betadex Sulfobutyl Ether Sodium, Crospovidone, Dextran 40, Glycerin, Pentetic Acid, Poloxamer 188, Polyethylene Glycol 300, Polyethylene Glycol 3350, Polyethylene Glycol 400, Polyethylene Glycol 4000, Polyethylene Glycol 600, Povidone, Povidone K12, Povidone K15, Propylene Glycol, Tartaric Acid (Granular), L-Cysteine Hydrochloride Monohydrate, Phosphoric Acid, Ammonium Sulphate, Propyl Paraben, Boric Acid, Sodium Metabisulfite Granular, Glycine, Ascorbic Acid, Benzoic Acid, Benzyl Alcohol, L-Arginine, Lactic Acid, Methyl Paraben, Polyoxyl 35 Castor Oil (Cremophor Elp), Polysorbate-80, Polysorbate-20, Potassium Phosphate Monobasic Crystal, Sodium Dihydrogen Phosphate Monohydrate, Soybean Oil, sodium acetate, Aspartic acid, glutamic acid and HCl. These components may be included individually or combined to promote overall stability of the formulation for long term storage.

The pH of the aqueous solution may be adjusted to the desired range by use of a pH adjusting agent. Examples of pH adjusting agent that may be used include buffering agents known in the art. The pH adjusting and/or buffering agent that may be used include, but are not limited to, glacial acetic acid, citric acid, sodium citrate, sodium hydroxide, hydrochloric acid, sulfuric acid, acetic acid, sodium acetate, tartaric acid, potassium hydroxide and the like and mixtures thereof. In one embodiment, the pH may be auto-adjusted to the desired range by the ingredients present in the solution. Preferably, the pH of the solution ranges from about 3.0 to about 5, in some embodiments about 3.5 to about 4.5, such as, 3.5, 3.6, 3.7, 3.8, 3.9, 4.0, 4.1, 4.2, 4.3, 4.4 or 4.5.

The disclosed stable parenteral dosage form comprise buffering agent in an amount ranging from about 0.001% w/v to about 50% w/v, from about 0.001% w/v to about 20.0% w/v, or from about 0.001% w/v to about 15.0% w/v, such as, for example, 0.001, 0.002, 0.003, 0.004, 0.005, 0.01, 0.1, 1.0, 1.2, 1.3, 1.4, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5 or 15% w/v. In one embodiment, the organic solvent is present in the disclosed ready-to-infuse aqueous solution in an amount ranging from about 0.001% w/v to about 5.0% w/v, and in certain embodiments, about 0.001-0.01% w/v.

In an embodiment, the disclosed dosage form may be stable with or without a buffer component, and may also be used as such without any additional pH adjustment requirement.

In one embodiment, the disclosed ready-to-infuse solution of oxytocin comprises glacial acetic acid or sodium hydroxide to adjust and maintain the pH in the range of about 3.0 to about 5.0. The disclosed ready-to-infuse aqueous solution is iso-osmolar to parenteral/plasma fluids. A tonicity adjusting agent or at least one osmogen that may be used may be selected from, but are not limited to, sucrose, mannitol, dextrose, sorbitol, glycerin, glycerol, sucrose, xylitol, fructose, mannose, maltitol, inositol, trehalose, sodium chloride, potassium chloride, calcium chloride, magnesium chloride, inorganic salts, urea and the like and mixtures thereof. In one embodiment, the at least one osmogen/tonicity adjusting agent used in the disclosed dosage form comprises sucrose, mannitol, dextrose, sodium chloride, sorbitol, or sodium acetate or a combination thereof.

In some embodiments, the disclosed infusion container of the parenteral dosage form of the present disclosure is a flexible infusion container, made up of a flexible material such as plastic or any other polymeric material. In other embodiments, the flexible infusion container may be an infusion bag, a flexible pouch, a soft bag, an infusion bottle or film, and the like. In another embodiment, the infusion container is a pre-filled syringe. The container may include one or more layers of such materials. Suitably, such materials may include, but are not limited to, polyolefin polymers, polyethylene, polypropylene; cyclo olefin polymers, cyclo olefin copolymers, polypropylene based polyolefin polymers; polycarbonates; modified polyolefin-polyethylene polymers or styrene-polyolefin based polymers or block co-polymers thereof. Preferably, the flexible infusion container is not impermeable in nature and possesses some permeation characteristics and the aqueous solution of oxytocin remains in contact with these materials of the container throughout the shelf life of the dosage form. In an embodiment, the dosage form is provided in an infusion bag.

In one embodiment, the flexible infusion containers are made of a material comprising a polymer of cyclic olefin such as cycloolefin homopolymer or cycloolefin copolymer or mixtures thereof. In a further embodiment, the container comprises an inner layer made of a cycloolefin polymer, a middle layer made up of a linear low density polyethylene polymer and an outer layer made of a low density polyethylene polymer. Such containers are available commercially, and have a water vapour transmission rate of 2 g/(m²/day) when measured at (40° C./90% relative humidity); an oxygen transmission rate of 570 ml/(m²·24 hour·atm) when measured at (23° C./0% relative humidity); and a carbon dioxide transmission rate of 3400 ml/(m²·24 hour·atm) when measured at 23° C./0% relative humidity.

In another embodiment, the flexible infusion container is made of an outer layer of polypropylene polymer, with styrene-ethylene-butylene (SEB) block copolymer and a middle and inner layer made of polypropylene based polyolefin polymer, with styrene-ethylene butylene block copolymer. Such containers are available commercially and have a water vapour transmission rate of 0.62 g/(m²/day) when measured at 23° C./60% relative humidity; an oxygen permeability of 1110 ml/(m²·24 hour·atm) when measured at 23° C./40% relative humidity; and a carbon dioxide transmission rate of 5149 ml/(m²·24 hour·atm). Alternatively, the flexible container is made of a multilayer polyolefin film having layers from outside to inside made up of CPET-Tie-PE-Tie-EPC. These containers generally have a water vapour transmission rate of 5.0 g/(m²/day) when measured at 38° C./100% relative humidity; an oxygen transmission rate of 1315 cm³/(m²·24 hour·atm) when measured at 73° F./0% relative humidity; and a carbon dioxide transmission rate of 3945 cm³/(m²·24 hour·atm).

In another embodiment, the infusion containers include an infusion port, which may act as an infusion connector having three assembled parts, including a central stopper made of chlorobutyl or bromobutyl rubber (latex free); an upper breakable part and a bottom part, both made of polycarbonate. In one embodiment, the infusion container contains a delivery port end for insertion of an infusion set cannula/needle. In an embodiment, the infusion container/bag and the delivery port connecting to the infusion needle form a system whereby during administration of the solution to the patient the vacuum created by outgress of solution is accommodated by the elasticity or flexibility of the infusion bag instead of ingress of external non-sterile air. The dosage form can advantageously maintain the sterility of the solution until it reaches the patient.

In one embodiment, the flexible infusion container includes a thermally resealable portion that is fusible in response to thermal energy, and a container body having a sealed empty chamber in fluid communication with the resealable portion for receiving therein the aqueous solution of the present invention. The method of filling the container includes penetrating the resealable portion with an injection member and introducing the aqueous solution of the present invention into the chamber, withdrawing the injection member while engaging the base of the body to substantially prevent axial movement of the body, and applying thermal energy to the resealable portion to thermally fuse the penetrated region thereof. Such systems are elaborated in U.S. Pat. No. 7,992,597, which is incorporated herein by reference. Further, the flexible infusion container may include a chamber for receiving an aqueous solution of the disclosed dosage form and a thermoplastic portion in fluid communication with the chamber. The thermoplastic portion defines a penetrable region that is penetrable by a filling member and is heat resealable to hermetically seal an aperture therein by applying laser radiation at a predetermined wavelength and power and in a predetermined time period. Such systems are elaborated in U.S. Pat. No. 7,490,639, which is incorporated herein by reference.

In yet another embodiment, the flexible infusion container includes a sealed chamber; a first penetrable septum in fluid communication with the chamber that is formed of an elastic material and is penetrable by a first injection member to fill the first chamber with the aqueous solution of the disclosed dosage form there through; and a second penetrable septum movable between first and second positions. In the first position, at least a portion of the second septum is spaced away from the first septum to allow the injection member to penetrate the first septum and aseptically or sterile fill the chamber with the aqueous solution of the present invention there through. In the second position, the portion of the second septum overlies and seals a resulting injection aperture in the first septum after withdrawal of the first injection member therefrom, and is penetrable by a second injection member to penetrate the first and second septums and withdraw the filled aqueous solution of the present invention from the chamber and through the second injection member. Such systems are elaborated in United States Published Patent Application Number US20130333796, which is incorporated herein by reference.

In one embodiment, the infusion container is rigid and is made of a material such as glass. Such infusion containers include infusion vials, infusion bottles, or pre-filled syringes. However, in some further embodiments, the container does not have material that contains borate or boron.

In another embodiment, the container is a pre-filled syringe. The pre-filled syringe may be made of a material having at least one non-glass component. The barrel of the pre-filled syringe can be made of appropriate plastic or polymeric material. In one aspect, the syringe comprises a barrel made up of cyclic olefin polymer, cyclic olefin copolymer, polypropylene, polycarbonate and the like. The syringe may further comprise an elastomeric tip cap, made of material such as a chloro-butyl formulation. The syringe may comprise a plunger stopper made up of rubber material such as bromo-butyl rubber.

In one embodiment, the container may be further packaged in a secondary packaging. The secondary packaging may comprise a second container such as a pouch or overwrap or film or carton. The secondary packaging may further comprise an oxygen scavenger. In one embodiment, the secondary packaging is designed to protect the solution of oxytocin from light.

In some further embodiments, the secondary packaging pouch or film or overwrap or carton is made of a suitable light protective material such as aluminium. Non-limiting examples of material constituting secondary packaging or secondary containers include aluminum, various polymers and copolymers, such as polyamide, ethylene vinyl alcohol copolymer, and the like. Aluminum based containers may be preferred and include aluminium pouches, aluminium plated films, aluminium foils, aluminum laminate films, composite aluminum films co-extruded with other polymers such as polyethylene, polypropylene, EVA, EMA, EAA, etc. In one embodiment, the secondary container is an overwrap pouch made of a composite polymer aluminium film having PET, Nylon-6, aluminium foil, and CPP (polypropylene/ethylene block copolymer) from outside to inside, the layers being either co-extruded and/or fixed using an adhesive with the other layer. In yet another embodiment, the secondary container is an overwrap pouch made of PET/NY/Aluminum/Oxygen absorbing layer/Polyethylene. In another preferred embodiment, the second container is an overwrap pouch made up of PET/NY/Aluminum/Oxygen absorbing layer/Polypropylene. In another preferred embodiment, the second container is an overwrap pouch made up of PET/NY/AL/OA/CPP. In some embodiments, the dosage form may further comprise an oxygen scavenger, which may be placed in between the infusion container and the second overwrap container or in some embodiments, the overwrap pouch may have a layer of oxygen absorbing material which acts as an oxygen scavenger, such as fused silica bags or iron containing adsorbents, such as iron oxide and the like. The oxygen scavenger or oxygen scavenging layer material may be a suitable material capable of quickly absorbing oxygen and having good oxygen absorbing capacity and heat resistance. Non-limiting examples of such oxygen scavenging materials include, but are not limited to, iron, silica, charcoal, and the like. Preferably, the oxygen scavenging material is an iron based material. In one embodiment, the oxygen scavenger may be an iron based self-reacting type or iron based water dependent type oxygen scavenger/absorber. In one embodiment, the space between the infusion container and secondary overwrap container or pouch is filled with an inert gas such as nitrogen or argon.

The parenteral dosage form of the present invention is sterile. The term “sterile” or ‘sterilized’ as used herein, means that the aqueous solution has been brought to a state of sterility and has not been subsequently exposed to microbiological contamination, i.e., the sterility of the solution present in the container has not been compromised. The solution complies with the sterility requirements of standard Pharmacopoeias, such as the United States Pharmacopoeias (USP). Sterilization may be achieved by suitable techniques such as filtration sterilization, radiation sterilization, and the like.

In one embodiment, the present disclosure provides a process for preparation of a stable, ready-to-infuse parenteral dosage form, wherein said process comprising: a) taking water for injection in an amount of about 60 to 80% of the batch size in a container; b) purging nitrogen to reduce dissolved oxygen level below 1 ppm; c) adding a suitable excipient to the container and dissolving; d) checking the pH and noting the values followed by adding and dissolving of an osmogen; e) checking the pH and noting the values followed by adding another osmogen, if necessary, and dissolving under stirring; f) checking the pH and noting the value and if required adjusting the pH of the bulk to a pH of 3.0-5.0 by gradual addition of a suitable pH adjusting agent; g) adding a desired strength of oxytocin or pharmaceutically acceptable salt stock with continuous nitrogen purging, then adjusting the volume to final batch size using water for injection and mixing well; h) filtering the solution by using 0.2 micron membrane filter; and (i) filling in a flexible infusion bag or container with continuous nitrogen purging during the course of the process to keep dissolved oxygen level below 1 ppm and stoppering the bag/container.

In another embodiment, the bag may be overwrapped using a suitable aluminium pouch, optionally with an oxygen scavenger and nitrogen gas.

In one embodiment, the disclosure provides a method for initiation or improvement of uterine contractions antepartum, where this is desirable and considered suitable for reasons of fetal or maternal concerns, in order to achieve vaginal delivery.

In yet another embodiment, the present disclosure provides methods for induction of labor in patients with a medical indication for the initiation of labor, such as Rh problems, maternal diabetes, preeclampsia at or near term, when delivery is in the best interests of mother and fetus or when membranes are prematurely ruptured and delivery is indicated, by administering a parenteral dosage form of oxytocin comprising a ready-to-infuse, stable aqueous solution of oxytocin or a pharmaceutically acceptable salt thereof.

In another embodiment the present disclosure provides methods for stimulation or reinforcement of labor, as in selected cases of uterine inertia, by administering a parenteral dosage form of oxytocin comprising a ready-to-infuse, stable aqueous solution of oxytocin or a pharmaceutically acceptable salt thereof.

In one embodiment the present disclosure provides methods for using the parenteral dosage form of oxytocin or a pharmaceutically acceptable salt thereof comprising a ready-to-infuse, stable aqueous solution of oxytocin or a pharmaceutically acceptable salt thereof as an adjunctive therapy in the management of incomplete or inevitable abortion. In the first trimester, curettage is generally considered primary therapy.

In a further embodiment, the present disclosure provides a method for second trimester abortion, wherein oxytocin infusion will often be successful in emptying the uterus.

In another embodiment, the present disclosure provides a method for postpartum uterine contractions during the third stage of labor and to control postpartum bleeding or hemorrhage.

In another embodiment, the disclosure provides use of the disclosed stable parenteral dosage form of oxytocin or a pharmaceutically acceptable salt thereof comprising a ready-to-infuse aqueous solution of oxytocin or a pharmaceutically acceptable salt thereof for:
(1) antepartum initiation, or improvement of uterine contractions, where this is desirable and considered suitable for reasons of fetal or maternal concern, in order to achieve vaginal delivery;
(2) induction of labor in patients with a medical indication for the initiation of labor, such as Rh problems, maternal diabetes, preeclampsia at or near term, when delivery is in the best interests of mother and fetus or when membranes are prematurely ruptured and delivery is indicated;
(3) stimulation or reinforcement of labor, as in selected cases of uterine inertia; and
(4) as adjunctive therapy in the management of incomplete or inevitable abortion.

In another embodiment, the disclosure provides use of a stable parenteral dosage form comprising, a ready-to-infuse aqueous solution of oxytocin or a pharmaceutically acceptable salt thereof, for second trimester abortion, wherein the dosage form is useful for emptying the uterus.

In another embodiment, the disclosure provides use of the disclosed stable parenteral dosage form of oxytocin or a pharmaceutically acceptable salt thereof comprising a ready-to-infuse aqueous solution of oxytocin or a pharmaceutically acceptable salt thereof for inducing Postpartum uterine contractions during the third stage of labor and to control postpartum bleeding or hemorrhage.

In yet another embodiment, the disclosure provides a stable, ready-to-infuse, aqueous parenteral dosage form comprising oxytocin or its pharmaceutically acceptable salt, a pH adjusting agent to provide a pH in the range of about 3.0 to about 5.0, and at least one pharmaceutically acceptable osmogen, for use in initiation or improvement of uterine contractions, during vaginal child birth or delivery.

In another embodiment, the disclosure provides a stable, ready-to-infuse, aqueous parenteral dosage form comprising oxytocin or its pharmaceutically acceptable salt, a pH adjusting agent to provide a pH in the range of about 3.0 to about 5.0, at least one pharmaceutically acceptable osmogen and an infusion container filled with the said aqueous solution, for Antepartum use in induction of labor in patients with a medical indication for the initiation of labor, such as Rh problems, maternal diabetes, preeclampsia at or near term, when delivery is in the best interests of mother and fetus or when membranes are prematurely ruptured and delivery is indicated; or for stimulation or reinforcement of labor, as in selected cases of uterine inertia; or as adjunctive therapy in the management of incomplete or inevitable abortion.

In the context of this specification “comprising” is to be interpreted as “including”. Aspects of the invention comprising certain elements are also intended to extend to alternative embodiments “consisting” or “consisting essentially” of the relevant elements.

The term “patient” may refer to a human patient.

Where technically appropriate, embodiments of the invention may be combined. Embodiments are described herein as comprising certain features/elements. The disclosure also extends to separate embodiments consisting or consisting essentially of said features/elements.

Any embodiments specifically and explicitly recited herein may form the basis of a disclaimer either alone or in combination with one or more further embodiments.

Hereinafter, the invention will be more specifically described by way of Examples. The examples are not intended to limit the scope of the invention and are merely used as illustrations.

### EXAMPLES

### Example-1

Various sample batches were prepared to evaluate the effect of various excipients alone or in combination on the stability of a dosage form as shown in Table-1:

TABLE 1
Sample compositions with various excipients

| | Batch No | | | | | | | | | | |
|---|---|---|---|---|---|---|---|---|---|---|---|
| | A | B | C | D | E | F | G | H | I | J | K |
| Component | Each mL contains | | | | | | | | | | |
| Oxytocin | 0.06 | 0.06 | 0.06 | 0.06 | 0.06 | 0.06 | 0.06 | 0.06 | 0.06 | 0.06 | 0.06 |
| (USP) | | | | | | | | | | | |
| Sodium | — | — | — | — | 1 | 1 | 1 | 1 | — | — | 0.5 |
| Acetate | | | | | mg | mg | mg | mg | | | mg |
| NaCl | 9 | — | — | 2.5 | 4.5 | — | — | — | — | — | 2 |
| | mg | | | mg | mg | | | | | | mg |
| Dextrose | — | 50 | — | — | — | 50 | — | — | 20 | — | — |
| | | mg | | | | mg | | | mg | | |
| Mannitol | — | — | 50 | — | — | — | 45 | — | — | 20 | — |
| | | | mg | | | | mg | | | mg | |
| Sucrose | — | — | — | 55 | — | — | — | 55 | 55 | 55 | 50 |
| | | | | mg | | | | mg | mg | mg | mg |
| Glacial | q.s. to | q.s. to | q.s. to | q.s. to | q.s. to | q.s. to | q.s. to | q.s. to | q.s. to | q.s. to | q.s. to |
| Acetic acid | pH | pH | pH | pH | pH | pH | pH | pH | pH | pH | pH |
| | 4.0 | 4.0 | 4.0 | 4.0 | 4.0 | 4.0 | 4.0 | 4.0 | 4.0 | 4.0 | 4.0 |
| Water for | q.s | q.s. | q.s | q.s | q.s | q.s | q.s | q.s | q.s | q.s | q.s |
| injection | | | | | | | | | | | |

Manufacturing Procedure:
I. Preparation of Bulk Solution and Filling:
   i. Weighed quantity of water for injection (60-80% of batch size) was collected in a container, and nitrogen was purged to reduce the dissolved oxygen (DO) level below 1 ppm;
   ii. Accurately weighed quantity of sodium acetate (if part of the composition) was added and dissolved completely, followed by measuring the pH of the solution;
   iii. Accurately weighed quantity of osmogen (sodium chloride, dextrose, mannitol, or sucrose) was added and dissolved under stirring respectively as in above table, with measurement of the pH;
   iv. The pH of the solution was adjusted to 4.0 using gradual addition of glacial acetic acid (10%) or sodium hydroxide (10%);
   v. Calculated quantity of oxytocin stock was added with continuous nitrogen purging;
   vi. Volume was made up to the final batch size with water for injection and mixed well under stirring;
   vii. The solution was aseptically filtered through a 0.2 μm filter and filled into various packing materials (different infusion bags and vials);
   viii. Nitrogen was continuously purged during the course of the process to maintain the DO level below 1 ppm with continuous nitrogen purging; and
   ix. Stoppered bag/vials immediately.
II. Overwrap: Infusion bags were overwrapped with nitrogen blanketing and an oxygen scavenger.
   The sample compositions were stored at 2-8° C., 25° C./40% RH, 30° C./65% RH; 40° C./25% RH for stability study duration.

TABLE 2
Initial Stability data of the sample formulations from
Table-1, in different packing materials coded as B1-B5:

| | | Batch No | | | | | | | | | | |
|---|---|---|---|---|---|---|---|---|---|---|---|---|
| Packing | | A | B | C | D | E | F | G | H | I | J | K |
| Material | | % Assay & pH Values | | | | | | | | | | |
| B-1 | Assay | 94.88 | 94.6 | 82.7 | 97.0 | 90.41 | 104.5 | 101.6 | 101.5 | 103.4 | 100.7 | 98.35 |
| | pH | 3.99 | 4.13 | 4.47 | 3.92 | 3.97 | 4.13 | 4.13 | 4.13 | 4.44 | 4.25 | 4.04 |
| B-2 | Assay | 93.1 | 91.2 | 84.9 | 98.0 | 95.4 | 107.5 | 98.7 | 100.4 | 105.9 | 101.4 | 97.3 |
| | pH | 4.07 | 4.11 | 4.49 | 4.01 | 4.01 | 4.14 | 4.14 | 4.13 | 4.5 | 4.25 | 4.04 |
| B-3 | Assay | 66.64 | 74.2 | 70.2 | 95.9 | 63.84 | 83.7 | 90.7 | 101.6 | 102.0 | 100.0 | 95.76 |
| | pH | 3.95 | 4.13 | 4.59 | 3.86 | 3.96 | 4.13 | 4.15 | 4.13 | 4.53 | 4.26 | 4.02 |
| B-4 | Assay | 62.4 | 64.8 | 66.6 | 95.9 | 46.7 | 82.7 | 82.5 | 101.5 | 100.1 | 97.76 | 94.9 |
| | pH | 4.08 | 4.2 | 4.57 | 3.51 | 4.02 | 4.13 | 4.14 | 4.14 | 4.56 | 4.27 | 4.06 |
| B-5 | Assay | 17.6 | 58.6 | 70.0 | 78.8 | 50.6 | 71.9 | 70.7 | 98.85 | 93.3 | 97.77 | 78.6 |
| | pH | 4.07 | 4.24 | 4.61 | 3.95 | 4.04 | 4.12 | 4.14 | 4.11 | 4.54 | 4.29 | 4.08 |

TABLE 2-1
Packaging material description:

| Sr. No. | Code | Description | Technical Description |
|---|---|---|---|
| 1 | B1 | 100 ml vial | Type 1 USP glass vial |
| 2 | B1' | 50 ml vial | Type 1 USP glass vial |
| 3 | B2 | Bag | Copolyester/Ethylene Methyl |
| | | | Acrilate/Polyethylene based |
| | | | polymer/Polyethylene based |
| | | | polymer/Polyolefin copolymer |
| | | | (Outer layer/tie layer/middle |
| | | | layer/tie layer/inner layer) |
| 4 | B3 | Bag | PP/LLDPE/COP (Outside/middle/inner) |
| 5 | B4 | Bag | HDPE/LLDPE/HDPE |
| | | | (Outside/middle/inner) |
| 2 | B5 | Bag | VMPET/ad/(LLDPE/tie Active |
| | | | Barrier/tie/LLDPE)/ad/(HDPE/ |
| | | | LLDPE/COP/LLDPE/HDPE) |

PP: Polypropylene
LLDPE: Linear low-density polyethylene
LDPE: Low density polyethylene
HDPE: High-density polyethylene
VMPET: Vacuum Metalized Polyethylene Terephthalate
COP: cyclo-olefin co-polymer
Tie: Modified polyolefin
Ad: adhesive

Observation:
Samples with different osmogens showed different assay values in the initial samples depicting their stability was impacted by the osmogen used in the ready-to-infuse dosage form. Further, the type of packing also played a role in the stability of the formulations.
Samples containing sucrose were found to be more stable in all the packing materials.
In addition to various osmogens, the presence of acetate buffers also showed significant impact on the stability of the ready-to-infuse dosage form. The dosage forms with sucrose and sodium acetate were found to be more stable.
Additionally, the addition of sucrose to another osmogen in a formulation was found to improve stability.

### Example 2

The sample formulations shown in below Table-3 were prepared as described below.

TABLE 3
Composition of Oxytocin RTU (Ready to use):

| | Batch No | P-1 | | P-2 | | P-3 | |
|---|---|---|---|---|---|---|---|
| Sr. No | Components | Each mL contains | | | | | |
| 1 | Oxytocin | 0.06 | IU | 0.06 | IU | 0.06 | IU |
| 2 | Sodium acetate | 1 | mg | 1.5 | mg | 0.5 | mg |
| 3 | Sucrose | 55 | mg | 55 | mg | 55 | mg |

| | | | | | |
|---|---|---|---|---|---|
| 4 | Mannitol | — | — | 10 | mg |

| | | | | |
|---|---|---|---|---|
| 5 | Glacial acetic | q.s to pH 4 | q.s to pH 4 | q.s to pH 4 |
| | acid/NaOH | | | |

| | | |
|---|---|---|
| 6 | Water for Injection | Qs |

Manufacturing Process:
I. Preparation of bulk solution and filling
i. Weighed quantity of water for injection (60-80% of batch size) was collected in a container and nitrogen was purged to reduce the dissolved oxygen level below 1 ppm;
ii. Accurately weighed quantity of sodium acetate was added and dissolved, and the pH was measured;
iii. Accurately weighed quantity of sucrose was added and dissolved under stirring, and the pH was measured;
iv. Accurately weighed quantity of mannitol (as in Table-3) was added and dissolved under stirring, and pH was measured;
v. The pH of the solution was adjusted to 4.0 by gradual addition of glacial acetic acid (10%) or sodium hydroxide (10%);
vi. Calculated quantity of oxytocin stock (0.01 mg/mL) was added with continuous nitrogen purging;
vii. Volume was made up to the final batch size with water for injection and mixed well under stirring;
viii. The solution was aseptically filtered through a 0.2 μm filter and filled into various packing materials (infusion bags and vials);
ix. Nitrogen was continuously purged during the course of the process to maintain the dissolved oxygen level below 1 ppm with continuous nitrogen purging; and
x. Stoppered the bags/vials immediately.
II. Overwrap: The infusion bags were overwrapped with nitrogen blanketing and an oxygen scavenger.

TABLE 4
Stability of various formulations from Table-3 batch P1 and
P1' (composition same as P1), were evaluated in different packing
materials under both cold and room temperature
conditions as shown in below table:

| Batch No-> | | P1' | | P1 | |
|---|---|---|---|---|---|
| Stage | | Assay | pH | Assay | pH |
| Packing Material | | 90-110% | 3-5 | 90-110% | 3-5 |
| B1' | Initial | 105.48 | 4.08 | — | — |
| | 2-8° C. 3M | 106.97 | 4.1 | — | — |
| | 2-8° C. 6M | 107.9 | 4.09 | — | — |
| | 2-8° C. 9M | 106.99 | 4.07 | — | — |
| | 2-8° C. 12M | 105.66 | 4.07 | — | — |
| | 25° C./40% RH-1M | 106.58 | 4.03 | | |
| | 25° C./40% RH-2M | 105.64 | 4.03 | — | — |
| | 25° C./40% RH-3M | 105.18 | 4.04 | — | — |
| | 25° C./40% RH-6M | 106.36 | 4.08 | — | — |
| | 25° C./40% RH-9M | — | — | — | — |
| B1 | Initial | 106.53 | 4.08 | 101.48 | 4.13 |
| | 2-8° C. 3M | 106.57 | 4.09 | 101.81 | 4.17 |
| | 2-8° C. 6M | 106.9 | 4.10 | 100.65 | 4.06 |
| | 2-8° C. 9M | 106.63 | 4.07 | 101.06 | 4.13 |
| | 2-8° C. 12M | 105.73 | 4.07 | 98.03 | 4.13 |
| | 2-8° C. 21M | — | — | 96.06 | 4.15 |
| | 2-8° C. 24M | 104.75 | 4.09 | — | — |
| | 25° C./40% RH-1M | 106.69 | 4.03 | 96.38 | 4.11 |
| | 25° C./40% RH-2M | 105.62 | 4.03 | 97.81 | 4.16 |
| | 25° C./40% RH-3M | 105.04 | 4.05 | 99.73 | 4.16 |
| | 25° C./40% RH-6M | 104.62 | 4.09 | 95.67 | 4.05 |
| | 25° C./40% RH-9M | — | — | 92.12 | 4.11 |
| B3 | Initial | 105.35 | 4.08 | 101.61 | 4.13 |
| | 2-8° C. 3M | 106.63 | 4.07 | 100.79 | 4.15 |
| | 2-8° C. 6M | 107.9 | 4.1 | 97.99 | 4.06 |
| | 2-8° C. 9M | 105.39 | 4.07 | 102.69 | 4.11 |
| | 2-8° C. 12M | 102.53 | 4.07 | 98.36 | 4.13 |
| | 2-8° C. 21M | — | — | 97.23 | 4.16 |
| | 2-8° C. 24M | 105.69 | 4.09 | — | — |
| | 25° C./40% RH-1M | 107.35 | 4.03 | 99.5 | 4.12 |
| | 25° C./40% RH-2M | 105.93 | 4.04 | 98.84 | 4.17 |
| | 25° C./40% RH-3M | 104.85 | 4.06 | 98.91 | 4.15 |
| | 25° C./40% RH-6M | 103.56 | 4.10 | 95.65 | 4.06 |
| | 25° C./40% RH-9M | — | — | 93.51 | 3.98 |
| B4 | Initial | 104.12 | 4.08 | 101.51 | 4.14 |
| | 2-8° C. 3M | 104.54 | 4.07 | 99.67 | 4.18 |
| | 2-8° C. 6M | 105.76 | 4.1 | 97.97 | 4.08 |
| | 2-8° C. 9M | 106.57 | 4.07 | 98.78 | 4.01 |
| | 2-8° C. 12M | 106.18 | 4.07 | 95.51 | 4.13 |
| | 2-8° C. 21M | — | — | 95.25 | 4.15 |
| | 2-8° C. 24M | 105.03 | 4.08 | — | — |
| | 25° C./40% RH-1M | 104.76 | 4.02 | 98.25 | 4.12 |
| | 25° C./40% RH-2M | 102.58 | 4.04 | 99.21 | 4.15 |
| | 25° C./40% RH-3M | 104.38 | 4.06 | 95.05 | 4.18 |
| | 25° C./40% RH-6M | 105.25 | 4.09 | 93.57 | 4.09 |
| | 25° C./40% RH-9M | — | — | 101.47 | 3.97 |
| B5 | Initial | 104.84 | 4.07 | 98.85 | 4.11 |
| | 2-8° C. 3M | 106.18 | 4.06 | 100.88 | 4.17 |
| | 2-8° C. 6M | 106.78 | 4.10 | 97.70 | 4.09 |
| | 2-8° C. 9M | 103.64 | 4.07 | 96.24 | 3.97 |
| | 2-8° C. 12M | 104.9 | 4.07 | 97.14 | 4.13 |
| | 2-8° C. 21M | — | — | 96.99 | 4.16 |
| | 2-8° C. 24M | 104.06 | 4.09 | — | — |
| | 25° C./40% RH-1M | 105.97 | 4.04 | 97.83 | 4.12 |
| | 25° C./40% RH-2M | 103.82 | 4.02 | 97.35 | 4.17 |
| | 25° C./40% RH-3M | 105.27 | 4.06 | 99.11 | 4.17 |
| | 25° C./40% RH-6M | 103.09 | 4.11 | — | — |

Packaging Material Code:-as in Table 2.1

Observation:
All the samples in both the P1 and P1′ batches were found to be stable for a longer period of time under both stages of 2-8° C. and 25° C./40% RH for at least 6 months in tested packing materials without any significant change in pH and assay values from the initial samples.

### Example 3

Various sample batches were prepared to evaluate the effect of the excipients combination on the stability of a dosage form as shown in the below tables:

TABLE 5
Sample composition with various excipients:

| | Batch No | | | | | | |
|---|---|---|---|---|---|---|---|
| | 3A | 3B | 3C | 3D | 3E | 3F | 3G |
| Components | Each mL contains | | | | | | |
| Oxytocin | 0.06 | 0.06 | 0.06 | 0.06 | 0.06 | 0.06 | 0.06 |
| (USP) in IU | | | | | | | |
| Sodium | 1 | 1 | 1 | 1 | 1 | 1 | 0.1 |
| Acetate | mg | mg | mg | mg | mg | mg | mg |
| Mannitol | 50 | 50 | 50 | 50 | 50 | 50 | 50 |
| | mg | mg | mg | mg | mg | mg | mg |
| Glycine | — | 1 | — | — | — | — | — |
| | | mg | | | | | |
| HPBCD | — | — | 1 | 0.1 | 0.25 | 0.5 | 0.1 |
| | | | mg | mg | mg | mg | mg |
| Glacial Acetic | q.s | q.s | q.s | q.s | q.s | q.s | q.s |
| acid/NaOH | | | | | | | |
| Water for | q.s | q.s. | q.s | q.s | q.s | q.s | q.s |
| injection | | | | | | | |

Manufacturing Procedure:
I. Preparation of bulk solution and filling:
i. Weighed quantity of water for injection (60-80% of batch size) was collected in a container, and nitrogen was purged to reduce the dissolved oxygen level below 1 ppm;
ii. Accurately weighed quantity of sodium acetate (was added and dissolved completely) followed by measuring pH of the solution;
iii. Accurately weighted quantity of osmogen was added and dissolved under stirring respectively as in above table, with pH checking and noting;
iv. Accurately weighted quantity of excipient (glycine/HPBCD) was added and dissolved under stirring respectively as in above table, with pH checking and noting;
v. pH of the bulk was adjusted to 4.0 using gradual addition of glacial acetic acid (10%) or sodium hydroxide (10%);
vi. Calculated quantity of oxytocin stock was added with continues nitrogen purging.
vii. Volume was made up to final batch size with water for injection and mixed well under stirring;
viii. Solution was aseptically filtered through 0.2 μm filter and filled in to various packing materials (different infusion bags & vials);
ix. Nitrogen was continuously purged during the course of the process to maintain the DO level below 1 ppm with continue nitrogen purging; and
ix. Stoppered bag/vials immediately.
II. Overwrap: Infusion bags were overwrapped with nitrogen blanketing and an oxygen scavenger.

TABLE 6
Stability of various formulations from Table-5 were evaluated after storing
at 25° C./40% RH, in different packing materials as shown in below table:

| | | | Batch No | | | | | | |
|---|---|---|---|---|---|---|---|---|---|
| Packing | | Time | 3A | 3B | 3C | 3D | 3E | 3F | 3G |
| Material | | point | % Assay & pH Values | | | | | | |
| B1 | Assay | Initial | 98.81 | 98.86 | 101.34 | 99.99 | 100.8 | 100.6 | 99.52 |
| | | 25° C. 1 M | NP | NP | 99.69 | 96.6 | 96.48 | 96.84 | 100.33 |
| | | 25° C. 3 M | NP | NP | 101.77 | 96.18 | 95.85 | 96.4 | 103.32 |
| | pH | Initial | 3.95 | 3.99 | 3.96 | 3.97 | 3.96 | 3.98 | 3.91 |
| | | 25° C. 1 M | NP | NP | 3.95 | 4.0 | 3.99 | 4.01 | 3.94 |
| | | 25° C. 3 M | NP | NP | 3.92 | 3.92 | 3.87 | 3.91 | 3.95 |
| B4 | Assay | Initial | 88.27 | 87.32 | 100.91 | 99.38 | 99.73 | 100.58 | NP |
| | | 25° C. 1 M | NP | NP | 99.3 | 96.13 | 96.33 | 96.79 | NP |
| | | 25° C. 3 M | NP | NP | 98.9 | 95.12 | 95.51 | 96.39 | NP |
| | pH | Initial | 3.96 | 3.99 | 3.95 | 3.97 | 3.96 | 3.99 | NP |
| | | 25° C. 1 M | NP | NP | 3.96 | 4.0 | 3.99 | 4.01 | NP |
| | | 25° C. 3 M | NP | NP | 3.93 | 3.91 | 3.89 | 3.93 | NP |
| B5 | Assay | Initial | 84.26 | 86.94 | 101.3 | 99.61 | 100.2 | 99.91 | 100.47 |
| | | 25° C. 1 M | NP | NP | 100.33 | 95.94 | 96.36 | 96.91 | 97.91 |
| | | 25° C. 3 M | NP | NP | 99.6 | 95.76 | 96.21 | 96.48 | 99.75 |
| | pH | Initial | 3.96 | 4.0 | 3.95 | 3.97 | 3.96 | 3.99 | 3.92 |
| | | 25° C. 1 M | NP | NP | 3.95 | 4.01 | 3.99 | 4.0 | 3.93 |
| | | 25° C. 3 M | NP | NP | 3.92 | 3.90 | 3.88 | 3.92 | 3.96 |

Packaging Material Code: - as in Table 2.1

Observation:
Samples with different amounts of HPBCD showed different assay values depicting their stability was impacted by the HPBCD used in the ready-to-infuse dosage form, further the type of packing also played a role in the stability of formulation.
Sample containing mannitol only was relatively found to be unstable in all the infusion bags.
In addition to various excipients, the presence of different concentrations of acetate buffers also showed insignificant impact on the stability of the ready-to-infuse dosage forms so far, also the dosage forms with mannitol, HPBCD and sodium acetate were found to be relatively more stable.

### Example 4

Various sample batches were prepared to evaluate the effect of the excipients combination on the stability of dosage forms as shown in the below tables:

TABLE 7
Sample composition with various excipients:

| | Batch No | | | | | | | | | | |
|---|---|---|---|---|---|---|---|---|---|---|---|
| | 4A | 4B | 4C | 4D | 4E | 4F | 4G | 4H | 4I | 4J | 4K |
| Components | Each mL contains | | | | | | | | | | |
| Oxytocin | 0.06 | 0.06 | 0.06 | 0.06 | 0.06 | 0.06 | 0.06 | 0.06 | 0.06 | 0.06 | 0.06 |
| (USP) in IU | | | | | | | | | | | |
| Sodium | 1 | 1 | 1 | 1 | 1 | 0.75 | 0.5 | 0.25 | 0.1 | — | 0.1 |
| Acetate | mg | mg | mg | mg | mg | mg | mg | mg | mg | | mg |
| Sodium | 4.5 | 4.5 | 4.5 | 4.5 | 4.5 | 4.5 | 4.5 | 4.5 | 4.5 | 4.5 | 9 |
| Chloride | mg | mg | mg | mg | mg | mg | mg | mg | mg | mg | mg |
| HPBCD | — | 0.1 | 0.25 | 0.5 | 1 | 1 | 1 | 1 | 1 | 1 | 0.1 |
| | | mg | mg | mg | mg | mg | mg | mg | mg | mg | mg |
| Glacial Acetic | q.s | q.s | q.s | q.s | q.s | q.s | q.s | q.s | q.s | q.s | q.s |
| acid/NaOH | | | | | | | | | | | |
| Water for | q.s | q.s. | q.s | q.s | q.s | q.s | q.s | q.s | q.s | q.s | q.s |
| injection | | | | | | | | | | | |

Manufacturing Procedure:

Same procedure as set forth in Example 3 above. The osmogen used was sodium chloride and the excipient was as set forth in Table 7 above. Following preparation of bulk solution and filling the infusion bags were overwrapped with nitrogen blanketing and an oxygen scavenger.

TABLE 8
Stability of various formulations from Table-7 were evaluated after storing
at 25° C./40% RH, in different packing materials as shown in the below table:

| | | | Batch No | | | | | | | | | | |
|---|---|---|---|---|---|---|---|---|---|---|---|---|---|
| Packing | | | 4A | 4B | 4C | 4D | 4E | 4F | 4G | 4H | 4I | 4J | 4K |
| Material | | Stage | % Assay & pH Values | | | | | | | | | | |
| B-1 | Assay | Initial | 97.84 | 96.75 | 99.13 | 98.73 | 98.42 | 95.93 | 95.99 | 96.09 | 95.37 | 95.39 | 100.44 |
| | | 25° C._1 M | NP | 94.94 | 95.32 | 95.83 | 95.67 | 98.4 | 98.42 | 97.8 | 97.47 | 98.21 | 97.59 |
| | | 25° C._3 M | NP | 94.05 | 93.85 | 94.75 | 94.99 | 95.34 | 94.09 | 95.24 | 95.09 | 94.94 | 99.78 |
| | pH | Initial | 3.95 | 3.97 | 3.95 | 3.97 | 3.98 | 3.89 | 3.92 | 3.89 | 3.87 | 3.89 | 3.97 |
| | | 25° C._1 M | NP | 3.97 | 3.95 | 3.95 | 3.96 | 3.94 | 3.94 | 3.92 | 3.92 | 3.88 | 3.93 |
| | | 25° C._3 M | NP | 3.88 | 3.85 | 3.86 | 3.87 | 3.93 | 3.93 | 3.91 | 3.90 | 3.90 | 3.98 |
| B-4 | Assay | Initial | 80.85 | 95.72 | 98 | 97.42 | 98.08 | 95.98 | 95.95 | 95.67 | 95.49 | 97.13 | NP |
| | | 25° C._1 M | NP | 93.89 | 94.58 | 95.38 | 95.36 | 97.91 | 97.67 | 98.3 | 97.26 | 97.6 | NP |
| | | 25° C._3 M | NP | 93.14 | 93.68 | 94.4 | 94.7 | 95.44 | 95.59 | 94.98 | 95.04 | 95.37 | NP |
| | pH | Initial | 3.96 | 3.97 | 3.95 | 3.97 | 3.98 | 3.89 | 3.91 | 3.91 | 3.89 | 3.89 | NP |
| | | 25° C._1 M | NP | 3.98 | 3.95 | 3.96 | 3.96 | 3.92 | 3.95 | 3.93 | 3.87 | 3.90 | NP |
| | | 25° C._3 M | NP | 3.86 | 3.84 | 3.86 | 3.86 | 3.92 | 3.93 | 3.92 | 3.91 | 3.93 | NP |
| B-5 | Assay | Initial | 66.27 | 94.34 | 98.11 | 97.92 | 98.79 | 96.34 | 95.99 | 95.77 | 95.31 | 96.77 | 98.27 |
| | | 25° C._1 M | NP | 93.92 | 94.89 | 95.53 | 95.92 | 98.56 | 98.29 | 98.15 | 97.34 | 98.67 | 96.97 |
| | | 25° C._3 M | NP | 93.49 | 94.2 | 95.1 | 95.04 | 95.86 | 95.65 | 95.12 | 95.03 | 95.17 | 99.26 |
| | pH | Initial | 3.95 | 3.97 | 3.96 | 3.99 | 3.97 | 3.90 | 3.92 | 3.89 | 3.87 | 3.89 | 3.98 |
| | | 25° C._1 M | NP | 3.96 | 3.95 | 3.96 | 3.98 | 3.92 | 3.95 | 3.93 | 3.87 | 3.87 | 3.93 |
| | | 25° C._3 M | NP | 3.86 | 3.86 | 3.85 | 3.88 | 3.92 | 3.93 | 3.9 | 3.89 | 3.90 | 3.96 |

Packaging Material Code: - as in Table 2.1

Observation:
Samples with different amounts of HPBCD showed different assay values depicting their stability was impacted by the HPBCD used in the ready-to-infuse dosage form. Further, the type of packing also played role in the stability of the formulation.
Samples with different amounts of sodium acetate showed no difference on assay values in the ready-to-infuse dosage form.
Sample containing sodium chloride only was relatively found to be unstable, in all the infusion bags.

In addition to various excipients, the presence of different concentrations of acetate buffers also showed insignificant impact on stability of the ready-to-infuse dosage forms. Also, the dosage forms with sodium chloride, HPBCD and sodium acetate were found to be relatively more stable.

### Example-5

Various sample batches were prepared to evaluate the effect of the excipients combination and kept for stability evaluation in various types of packing materials in different temperature and humidity conditions.

TABLE 9
Sample composition with various excipients:

| Batch No | P-3 | 5A |
|---|---|---|
| Components | Each ml contains | |
| Oxytocin (USP) in IU | 0.06 | 0.06 |
| Sodium acetate | 0.5 mg | 1 mg |
| Sucrose | 55 mg | 20 mg |
| Mannitol | 10 mg | 20 mg |
| Glacial acetic acid/NaOH | q.s | q.s |
| Water for injection | q.s | q.s. |

Manufacturing Procedure:

Same procedure as set forth in Example 3. The osmogen used was sucrose and mannitol as set forth in Table 9 above. Following preparation of bulk solution and filling, the infusion bags were overwrapped with nitrogen blanketing and an oxygen scavenger.

TABLE 10
Stability of various formulations from Table-9 were
evaluated after storing at 25° C./40% RH, in different
packing materials as shown in the below table:

| Batch No | | | P-3 | 5A |
|---|---|---|---|---|
| | | Time point/ | % Assay & | |
| Packing Material | | Condition | pH Values | |
| B-1 | Assay | Initial | 96.71 | NP |
| | | 25° C._1M | 99.17 | NP |
| | | 25° C._3M | 96.36 | NP |
| | pH | Initial | 4.11 | NP |
| | | 25° C._1M | 4.15 | NP |
| | | 25° C._3M | 4.15 | NP |
| B-2 | Assay | Initial | 97.01 | NP |
| | | 25° C._1M | 99.63 | NP |
| | | 25° C._3M | 93.68 | NP |
| | pH | Initial | 4.09 | NP |
| | | 25° C._1M | 4.17 | NP |
| | | 25° C._3M | 4.16 | NP |
| B-3 | Assay | Initial | 96.98 | 97.04 |
| | | 25° C._1M | 98.32 | 97.67 |
| | | 25° C._3M | 99.58 | 99.63 |
| | pH | Initial | 4.11 | 4.06 |
| | | 25° C._1M | 4.18 | 3.93 |
| | | 25° C._3M | 4.16 | 4.08 |
| B-4 | Assay | Initial | 95.97 | 97.95 |
| | | 25° C._1M | 96.90 | 97.43 |
| | | 25° C._3M | 90.81 | 96.2 |
| | pH | Initial | 4.11 | 4.06 |
| | | 25° C._1M | 4.16 | 3.91 |
| | | 25° C._3M | 4.17 | 4.08 |
| B-5 | Assay | Initial | NP | 99.94 |
| | | 25° C._1M | NP | 96.25 |
| | | 25° C._3M | NP | 96.29 |
| | pH | Initial | NP | 4.06 |
| | | 25° C._1M | NP | 3.93 |
| | | 25° C._3M | NP | 4.08 |

Packaging Material Code :-as in Table 2.1

Observation:
Samples with higher amounts of sucrose with mannitol were relatively found to be stable, in all the infusion bags.

### Example-6

Various sample batches were prepared to evaluate the effect of the excipients combination and kept for stability evaluation in various types of packing materials in different temperature and humidity conditions.

TABLE 11
Sample composition with various excipients:

| Batch No | 6A | 6B |
|---|---|---|
| Components | Each ml contains | |
| Oxytocin (USP) | 0.06 | 0.02 |
| Sodium acetate | 0.01 mg | 0.01 mg |
| Sucrose | 90 mg | 90 mg |
| Glacial acetic acid/NaOH | q.s | q.s |
| Water for injection | q.s | q.s. |

Manufacturing Procedure:

Same procedure as set forth in Example 3. The osmogen used was sucrose. Following preparation of bulk solution and filling, the infusion bags were overwrapped with nitrogen blanketing and an oxygen scavenger.

TABLE 12
Stability of various formulations from Table-11, in different
packing materials coded as shown in the below table:

| Batch No | | | 6A | 6B |
|---|---|---|---|---|
| Packing Material | | Time point/Condition | % Assay & pH Values | |
| B-2 | Assay | Initial | 104.38 | 99.49 |
| | pH | Initial | 4.10 | 4.17 |
| B-3 | Assay | Initial | 102.12 | 99.32 |
| | pH | Initial | 4.17 | 4.17 |
| B-5 | Assay | Initial | 101.06 | 99.18 |
| | pH | Initial | 4.17 | 4.17 |

Observation:
Samples with higher amounts of sucrose were found to be satisfactory at initial in all the infusion bags.

1. A ready-to-infuse parenteral dosage form comprising a stable aqueous solution comprising:
a. oxytocin or a pharmaceutical acceptable salt thereof, and
b. a disaccharide;
wherein the ready-to-infuse parenteral dosage form is stable for at least 3 months.

2. The ready-to-infuse parenteral dosage form as claimed in claim 1, wherein the dosage form is stable at room temperature for at least 6 months.

3. The ready-to-infuse parenteral dosage form as claimed in claim 1, wherein the dosage form is stable at 2-8° C. for at least 6 months.

4. A ready-to-infuse parenteral dosage form comprising a stable aqueous solution comprising:
a. oxytocin or a pharmaceutical acceptable salt thereof, and
b. a disaccharide;
wherein the assay % of oxytocin in said solution is at least 90%.

5. The ready-to-infuse parenteral dosage form as claimed in claim 4, wherein the assay of oxytocin in the solution is within 90% to 110%.

6. The ready-to-infuse parenteral dosage form as claimed in claim 1, further comprising at least one osmogen.

7. The ready-to-infuse parenteral dosage form as claimed in claim 1, wherein the pH of the aqueous solution is in range of about pH 3.0 to about pH 5.0.

8. The ready-to-infuse parenteral dosage form as claimed in claim 1, wherein the level of any single known impurity in the solution is less than 3% by weight when stored at 2-8° C. or at 25° C./40% RH.

9. The ready-to-infuse parenteral dosage form as claimed in claim 1, wherein the level of total impurities in the solution is less than 16% by weight when stored at 2-8° C. or at 25° C./40% RH.

10. The ready-to-infuse parenteral dosage as claimed in claim 6, wherein the osmogen is selected from the group consisting of a sugar, sodium chloride, calcium chloride, magnesium chloride, potassium chloride, a sugar; other inorganic salts, urea or a mixture thereof.

11. The ready-to-infuse parenteral dosage form as claimed in claim 10, wherein the sugar is selected from a monosaccharide, disaccharide, polysaccharide or a sugar alcohol or a combination thereof.

12. The ready-to-infuse parenteral dosage form as claimed in claim 11, wherein the disaccharide is sucrose or a combination of monosaccharides, selected from sucrose, lactulose, lactose, maltose, trehalose, cellobiose, kojibiose, nigerose, isomaltose, sophorose, laminarbiose, gentiobiose, turanose, maltulose, palatinose, gentiobiulose, mannobiose, melibiose, melibulose, rutinose, rutinulose or xylobiose.

13. The ready-to-infuse parenteral dosage form as claimed in claim 11, wherein the sugar is selected from dextrose, glycerin, glycerol, sucrose, mannitol, xylitol, fructose, mannose, maltitol, inositol, trehalose or a combination thereof.

14. The ready-to-infuse parenteral dosage form as claimed in claim 13, wherein the sugar is a combination of sucrose and mannitol.

15. The ready-to-infuse parenteral dosage form as claimed in claim 1, wherein said dosage form is filled in an infusion container.

16. The ready-to-infuse parenteral dosage form as claimed in claim 15, wherein said infusion container is selected from an infusion bag, a perfusion bag, a flexible pouch, a soft bag, an infusion bottle or a pre-filled syringe.

17. The ready-to-infuse parenteral dosage as claimed in claim 15, wherein the infusion container further comprises an overwrap over the infusion container.

18. The ready-to-infuse parenteral dosage form as claimed in claim 17, wherein the overwrap comprises an aluminum pouch with an oxygen scavenger.

19. The ready-to-infuse parenteral dosage form as claimed in claim 1, wherein oxytocin or a pharmaceutically acceptable salt is present in a concentration range of about 0.005 IU per ml to about 10 IU per ml.

20. The ready-to-infuse parenteral dosage form as claimed in claim 6, wherein said at least one osmogen is present at a concentration range of about 1 mg/mL to about 110 mg/mL.

21. The ready-to-infuse parenteral dosage form as claimed in claim 11, wherein the sugar is present in a concentration ranging from about 5 mg/mL to about 90 mg/mL.

22. The parenteral dosage form as claimed in claim 1, wherein said aqueous solution is stable upon storage at 2-8° C. for at least 6 months, the level of any single impurity is less than 3% by weight and the level of total impurities is less than 10% by weight.

23. The parenteral dosage form as claimed in claim 1, wherein said aqueous solution is stable upon storage at 25° C./40% RH for at least 6 months, the level of any single impurity is not more than 3% by weight and the level of total impurities is not more than 16% by weight.

24. The parenteral dosage form as claimed in claim 6, wherein the aqueous solution comprises oxytocin or a pharmaceutical acceptable salt in a concentration range of about 0.005 to about 10 IU per mL, the at least one osmogen in a concentration range of about 1 to about 110 mg/mL, and the disaccharide is sucrose in a concentration range of about 5 to about 90 mg/mL and the pH of the solution is in range of pH 3.0 to 5.0.

25. The parenteral dosage form as claimed in claim 1, wherein the assay of oxytocin is within 90-110% for storage period of at least 6 months at room temperature and/or at 2-8° C.

26. A ready-to-infuse parenteral dosage form comprising a stable aqueous solution having a pH of 3.0 to 5.0 comprising:
(a) oxytocin or a pharmaceutical acceptable salt thereof in a concentration range of about 0.01 to about 10 IU per mL;
(b) a disaccharide;
(c) sodium acetate; and
(d) acetic acid/sodium hydroxide,
wherein (i) the solution is substantially free of chlorobutanol and dextrose and (ii) the oxytocin concentration, after storage at 2-8° C., is 90% or greater than the oxytocin concentration prior to storage.

27. The dosage form of claim 26, wherein the level of any single impurity in the solution is less than 3% by weight of the amount of oxytocin present prior to storage after storage at 2-8° C.

28. The dosage form of claim 26, wherein the solution is substantially free of a divalent metal salt and EDTA and salts thereof.

29. A ready-to-infuse parenteral dosage form comprising a stable aqueous solution having a pH of 3.0 to 5.0 comprising:
(a) oxytocin or a pharmaceutical acceptable salt thereof in a concentration range of about 0.01 to about 10 IU per mL;
(b) an excipient selected from sodium chloride, sucrose, mannitol, Hydroxypropyl Betadex (HPBCD or HP-β-CD) or mixtures thereof, wherein the mixture comprises at least two excipients,
wherein (i) the solution is substantially free of chlorobutanol and dextrose and (ii) the oxytocin concentration, after storage at 2-8° C., is 90% or greater than the oxytocin concentration prior to storage.

30. A method of inducing labor in a patient with a medical indication selected from Rh problems, maternal diabetes, preeclampsia comprising administering to the patient the parenteral dosage form according to claim 1.

31. (canceled)

32. A method for
(i) antepartum initiation, or improvement of uterine contractions to achieve vaginal delivery in a patient,
(ii) induction of labor in a patient with a medical indication for the initiation of labor;
(iii) stimulation or reinforcement of labor in a patient with uterine inertia; or
(iv) adjunctive therapy in the management of incomplete or inevitable abortion, the method comprising administering to the patient the parenteral dosage form according to claim 1.

33. A method for inducing Postpartum uterine contractions during the third stage of labor or to control postpartum bleeding or hemorrhage in a patient comprising administering to the patient the parenteral dosage form according to claim 1.
